(19) United States
(12) Patent Application Publication
(10) Pub. No.: US 2021/0214748 A1
(43) Pub. Date: Jul. 15, 2021

(54) VECTORS

(71) Applicant: AUTOLUS LIMITED, London (GB)

(72) Inventors: Simon Thomas, London (GB); Shimobi Onuoha, London (GB); Shaun Cordoba, London (GB)

(21) Appl. No.: 16/760,848

(22) PCT Filed: Oct. 31, 2018
(86) PCT No.: PCT/GB2018/053149

§ 371 Date: Apr. 30, 2020

(30) Foreign Application Priority Data
Nov. 1, 2017 (GB) 1718088.6

(51) Int. Cl. C12N 15/86; C07K 14/705

(52) U.S. Cl. CPC C12N 15/86; C07K 14/70596; C12N 2810/859; C12N 2810/852; C12N 2810/855; C12N 2800/40

(57) ABSTRACT

The present invention provides a kit of vectors comprising: (i) a first vector comprising a nucleic acid sequence encoding a first marker component; and (ii) a second vector comprising a nucleic acid sequence encoding a second marker component, wherein, when a cell is transduced with both the first and second vectors, the first and second marker components are expressed by the cell and associate forming a hetero-multimeric marker which is recognised by a cell sorting reagent whereas, when a cell is transduced with either the first or second vector alone, expression of the first or second marker component alone is not recognised by the cell sorting reagent.

The present invention relates to a kit of vectors. For example, retroviral vectors for transducing a cell.

In particular, the invention relates to a kit comprising a first vector comprising a nucleic acid encoding a first marker component, and a second vector comprising a nucleic acid encoding a second marker component. When a cell is transduced with both vectors, both marker components are expressed in the cell, and they associate to form a detectable hetero-multimeric marker.

The invention also relates to methods of making and detecting a cell transduced with such vectors, as well as pharmaceutical compositions and methods for treating/preventing a disease comprising administering compositions of such cells.

### BACKGROUND

Viral vectors have been used to transduce T-cells to express polypeptides of interest for decades. These vectors exploit the specialised molecular mechanisms evolved in viruses to efficiently transfer their genome inside the cell they infect. However, they have a finite transfer capacity that is generally considered to be around 8 to 10 kilobases (kb). The limit is due to the packaging efficiency being inversely proportional to the insert size.

Other potential non-viral mechanisms of T-cell-based gene therapy with a higher insert capacity are known, but these are often hampered by low efficiency of transduction and/or toxic effects that yield low T cell numbers.

In order to transduce a large insert size into a T cell whilst maintaining high efficiency, the genes encoded on a viral vector may be split into two or more separate vectors. Each vector is used to make the virus and all vectors are then pooled to transduce the cells. However, this multiple transduction approach often results in cells that are transduced with some but not all of the desired vectors. This leads to a non-uniform cell population comprising different vector integrants, which will not express all of the desired genes.

There is a need to provide improved methods of transducing and transfecting T cells with large insert sizes.

### DESCRIPTION OF THE FIGURES

FIG. 1—Schematic diagram illustrating a heterodimeric marker. The first marker component is a CD79a ectodomain and the second marker component is a fusion between CD79b ectodomain. and the transmembrane domain and a truncated endodomain from CD19. The first and second marker components associate via a di-sulphide bond.

FIG. 2—Schematic diagram illustrating another heterodimeric marker. The first marker component is a Kappa constant domain and the second marker component is a fusion between the CH1 domain from IgG1 and the transmembrane domain and a truncated endodomain from CD19. The first and second markers associate via at a di-sulphide bond.

FIG. 3—Schematic diagram illustrating a heterotrimeric marker. The first marker component is a Kappa constant domain; the second marker component is a fusion between the CD79b ectodomain and the transmembrane domain and a truncated endodomain from CD19; and the third marker is a fusion between CD79a ectodomain and a CH1 domain from IgG1. The first, second and third marker components associate via two di-sulphide bonds to form a hetero-trimeric marker.

FIG. 4—Amino acid sequences encoded by first and second vectors of a kit. The vectors encode first and second marker components which associate to form a hetero-dimeric marker as shown in FIG. 2.

FIG. 5—Amino acid sequences encoded by first, second and third vectors of a kit. The vectors encode first, second and third marker components which associate to form a hetero-trimeric marker as shown in FIG. 3.

FIG. 6A: A schematic diagram illustrating a first vector and a second vector, the first vector encoding a first chimeric antigen receptor (CAR), 2A peptide cleavage site and a first marker (CAR1-2A-Mα); and the second vector encoding a second CAR, 2A peptide cleavage site and a second marker (CAR2-2A-Mβ). The 2A peptide cleavage site is located between the marker and the CAR on each vector.

B: Schematic diagram illustrating the effect of transducing a cell with either or both vectors illustrated in FIG. 6A. When the cell is transduced with either the first or second vector alone, the transgene is expressed (either CAR1 or CAR2) but no detectable marker is expressed at the cell surface. When the cell is transduced with both first and second vectors, both CARs are expressed and association of the first and second marker components forms a stable hetero-dimeric marker, which is also expressed at the cell surface.

FIG. 7A: Schematic diagram of a first vector, second vector and third vector. The first vector encodes a first CAR, a2A peptide cleavage site and first marker (CAR1-2A-Mα); the second vector encodes a second CAR, a 2A peptide cleavage site and a second marker (CAR2-2A-Mβ); and the third vector encodes a third CAR, a 2A peptide cleavage site and a third marker (CAR3-2A-Mγ). The 2A peptide cleavage site is located between the marker and the CAR on each vector.

B: Schematic diagram illustrating the effect of transducing a cell with one, two or all three vectors shown in FIG. 7A. When the cell is transduced with one or two of the vectors the relevant transgene (i.e. CAR) is expressed but no detectable marker is expressed at the cell surface. All three of the vectors must be transduced for marker expression. Upon transduction of the first, second and third vectors, all three CARs are expressed and association of the first, second, and third marker components forms a stable hetero-trimeric marker which is expressed at the cell surface.

FIG. 8—Wild type and mutated signal sequences suitable for use to alter the relative of expression of marker components in a kit. One vector may encode the wild-type signal peptide sequence and the other vector may encode one of the altered sequences shown as “mutation 1” to “mutation 7”. The altered sequences are less efficient signal peptide, so the marker component encoded by the vector with the altered signal peptide will be expressed at a lower level in the cell than the marker component encoded by the vector with the wild type signal peptide. The relative expression of other transgene(s) on the same construct as the marker will be similarly affected, so that the polypeptide(s) of interest encoded by the vector with the altered signal peptide will be expressed at a lower level in the cell than the polypeptide of interest encoded by vector with the wild type signal peptide.

FIGS. 9 (A. and B.)—Surface expression of a heterodimer marker in 293 T cells (A.) and primary human T cells (B.) and a heterotrimer marker in 293T cells (C.).

A and B: 293T cells were either single transfected with each chain of the heterodimer marker (Vector 1 and Vector 2) or double transfected with both (Vector 1+Vector 2). Successful assembly of the heterodimer marker in double transfected cells was assessed by flow cytometry by staining with anti-human Kappa chain antibody, anti-human Fab antibody and using soluble CD19. A plasmid encoding for both chains of the heterodimer marker was used as a positive control. Both the 293K T cell and the primary human T-cell transduction results (using 4 healthy donor samples) demonstrate that selective expression of the heterodimer marker occurs only in double transduced T-cells with minimal background detected in single transduced T-cells.

C: 293T cells were single transfected with each chain of a heterotrimer marker (Vector 1, Vector 2 and Vector 3), or double transfected with Vector 1 and Vector 2 (Vector 1+Vector 2), or triple transfected with all three chains of the heterotrimer marker (Vector 1+Vector 2+Vector 3). Successful assembly of the heterotrimer marker was assessed by staining for soluble CD19 using flow cytometry. The results show that when a cell is transduced with one or two of the vectors no detectable marker is expressed at the cell surface. All three of the vectors must be transduced for marker expression.

### SUMMARY OF INVENTION

The inventors have developed a kit of vectors comprising nucleic acid sequences, each of which encode a marker component. The marker components stabilise upon association and form a detectable hetero-multimeric marker.

Thus, in a first aspect, the present invention provides a kit of vectors comprising:
(i) a first vector comprising a nucleic acid sequence encoding a first marker component; and
(ii) a second vector comprising a nucleic acid sequence encoding a second marker component.

When a cell is transduced with both the first and second vectors, the first and second marker components are expressed by the cell and associate forming a hetero-multimeric marker which is recognisable with an agent such as a cell sorting reagent.

When a cell is transduced with either the first or second vector alone, expression of the first or second marker component alone is not recognised by the cell sorting reagent.

The first marker component may be unstable when not associated with the second marker component. In this arrangement, the agent or cell sorting reagent may recognise the first marker component.

Alternatively, both the first and second marker components may be unstable when not associated. In this arrangement, the agent or cell sorting reagent may recognise either the first or second marker component.

The first marker component may be membrane-bound and the second marker component may be secreted in the absence of the first marker component. In this arrangement, the agent or cell sorting reagent may recognise the second marker component.

One marker component may comprise a Kappa constant domain and the other marker component may comprise the CH1 domain from IgG1.

One marker component may comprise a CD79a ectodomain and the other marker component may comprise a CD79b ectodomain.

The kit may comprise a third vector comprising a nucleic acid sequence encoding a third marker.

When a cell is transduced with the first, second and third vectors, the first, second and third marker components may be expressed by the cell and associate forming a hetero-multimeric marker which is recognised by a cell sorting reagent;

When a cell is transduced with one or two of the first, second or third vector(s), expression of one or two of the first, second or third marker component(s) may not be recognised by the cell sorting reagent.

The first, second and/or third marker component may be unstable when not associated as the heteromultimeric marker.

The first marker component may be membrane bound; the second marker component may be secreted in the absence of the first marker component; and the third marker component may be secreted unless the first and second marker components are also expressed. In this arrangement, the agent or cell sorting reagent may recognise the third marker component.

The first marker component may comprise a membrane-bound CD79a ectodomain; the second marker may comprise a CH1 domain from IgG1 and a CD79a ectodomain, and the third marker may comprise a Kappa constant domain.

At least one of the vectors in the kit of the first aspect of the invention may also comprise a nucleic acid sequence encoding a chimeric antigen receptor or a T cell receptor.

When the vectors in the kit are expressed inside a cell, the expression level of one marker component may be different to the expression level in the cell of another marker component.

The vectors encoding the two marker components may comprise different signal sequences.

In a second aspect, the present invention also provides a cell-surface hetero-multimeric marker for use in detecting a transduced cell population, wherein the hetero-multimeric marker comprises at least two marker components, the first marker component encoded by a nucleic acid sequence in a first vector and the second marker component encoded by a nucleic acid sequence in a second vector, wherein the first marker and second marker components associate.

The first and/or second marker component(s) may be unstable when not associated.

The second marker component may be secreted by the cell in the absence of the first marker component.

In a third aspect the present invention provides a cell which comprises a hetero-multimeric marker according to the second aspect of the invention and/or a cell transduced with a kit of vectors according to the first aspect of the invention.

The cell may be an immune cell such as a T cell or natural killer (NK) cell.

In a fourth aspect the present invention provides a method for making a cell according to the third aspect of the invention, which comprises the step of transducing or transfecting a cell with a kit of vectors according to the first aspect of the invention.

In a fifth aspect the present invention provides a method for preparing a composition of cells according to the third aspect of the invention which comprises the following steps:
(i) transducing or transfecting a cell sample with a kit of vectors according to the first aspect of the invention;
(ii) detecting expression of the hetero-multimeric marker using a an agent such as a cell-sorting reagent; and
(iii) selecting or sorting the detected cells to prepare a composition of cells which express the heteromultimeric marker.

The cell sorting reagent may be a soluble recombinant protein and the cells may be selected or sorted in step (iii) using a matrix which recognises the soluble recombinant protein.

The cell sorting reagent may be a fluorescently labelled soluble recombinant protein and the cells may be selected or sorted in step (iii) by flow cytometry.

The cell sorting reagent may be a soluble recombinant protein attached to a bead and the cells are selected or sorted in step (iii) by separation of the beads from the transduced/transfected cell sample.

In a sixth aspect the present invention provides a pharmaceutical composition comprising a plurality of cells according to the third aspect of the invention.

In a seventh aspect, the present invention provides a pharmaceutical composition according to the sixth aspect of the invention for use in treating and/or preventing a disease.

In an eighth aspect the present invention provides a method for treating and/or preventing a disease, which comprises the step of administering a pharmaceutical composition according to the sixth aspect of the invention to a subject.

The method may comprise the following steps:

I. isolation of a cell-containing sample from a subject
II. transducing or transfecting the cell-containing sample with a kit of vectors according to the first aspect of the invention
III. detecting expression of the hetero-multimeric marker using an cell-sorting reagent, thereby identifying a transduced/transfected cell population from the sample,
IV. selecting or sorting the cell population of (III) to achieve a purified subpopulation of transduced/transfected cells, and
V. administering the subpopulation of (IV) which express the hetero-multimeric marker to the subject.

In a ninth aspect the present invention provides the use of a pharmaceutical composition according to the sixth aspect of the invention in the manufacture of a medicament for the treatment and/or prevention of a disease.

The disease may be cancer.

The present invention provides a kit of vectors encoding marker components which stabilise upon association, forming a detectable hetero-multimeric marker capable of cell surface expression. When a cell is transduced with both or all vectors of the kit, the hetero-multimeric marker is expressed at the cell surface which is detectable with an agent such as a cell sorting reagent. However, when a cell is transduced with a subset of the vectors (for example one vector alone, or two out of three vectors) no marker is expressed at the cell surface which is detectable by the agent.

It is therefore possible to identify cells which have been transduced with all vectors in the kit by detection of expression of a single marker at the cell surface. This offers considerable advantages over methods involving detection of multiple markers (i.e. one for each vector) in terms of reduced complexity and better yield of cells following cell sorting.

It is possible to split large inserts between multiple vectors and select for cells transduced with all vectors using the principle of the invention which involves detection of a single heteromultimeric marker. It is therefore possible to increase the total insert size which can be transduced into a cell without increasing the complexity of the method for identifying and sorting for cells which contain all of the insert.

### DETAILED DESCRIPTION

### Kit of Vectors

The first aspect of the invention provides a kit of vectors. The kit of vectors comprises more than one vector. The kit of vectors comprises at least a first vector and a second vector, and in one embodiment, the kit of vectors comprises a first vector, a second vector and a third vector. The kit may contain, 2, 3, 4 or 5 vectors. The number of vectors in the kit of the present invention is related to the total size of the insert desired to be transduced into the host cell: where the total insert size is large, it may be split into a larger number of vectors.

The separate vectors in the kit of the present invention deliver separate nucleic acid sequences into the host cell such that, when a cell is transduced with all of the vectors of the kit, all of the desired polypeptides of interest (POI) are expressed by the cell. For example, the kit may express three POIs such as a first CAR, second CAR and suicide gene in the host cell.

Splitting the complete insert between multiple vectors offers advantages over a vector comprising a nucleic acid sequence encoding multiple polypeptides of interest within the same cassette. The single vector arrangement results in problems with efficiency of translation and transcription due to, for example, “promoter interference” whereby one promoter dominates and causes silencing of the second promoter. In addition, different promoters work differently in different cellular contexts and this makes consistent “tuning” of the relative expression of each transgene difficult to achieve.

### Vectors

The vectors may be viral vectors, such as retroviral vectors or lentiviral vectors.

The vector may be a plasmid.

The vectors may also be transposon based vectors or synthetic mRNA. The vectors may be capable of transfecting or transducing immune cells, such as a T cell or a NK cell.

### Retroviral Vectors

Retroviruses and lentiviruses may be used as a vector or delivery system for the transfer of a polypeptide of interest (POI), or a plurality of POIs, into a target cell. The transfer can occur in vitro, ex vivo or in vivo. When used in this fashion, the viruses are typically called viral vectors.

The POI may, for example, encode a T cell receptor or a chimeric antigen receptor (CAR) and/or a suicide gene.

Gamma-retroviral vectors, commonly designated retroviral vectors, were the first viral vector employed in gene therapy clinical trials in 1990 and are still one of the most used.

More recently, the interest in lentiviral vectors, derived from complex retroviruses such as the human immunodeficiency virus (HIV), has grown due to their ability to transduce non-dividing cells.

The most attractive features of retroviral and lentiviral vectors as gene transfer tools include the capacity for large genetic payload (up to about 8-10 kb), minimal patient immune response, high transducing efficiency in vivo and in vitro, and the ability to permanently modify the genetic content of the target cell, sustaining a long-term expression of the delivered gene.

The retroviral vector can be based on any suitable retrovirus that is able to deliver genetic information to eukaryotic cells. For example, the retroviral vector may be an alpharetroviral vector, a gammaretroviral vector, a lentiviral vector or a spumaretroviral vector. Such vectors have been used extensively in gene therapy treatments and other gene delivery applications.

The viral vector of the present invention may be a retroviral vector, such as a gamma-retroviral vector. The viral vector may be based on human immunodeficiency virus.

The viral vector of the present invention may be a lentiviral vector. The vector may be based on a non-primate lentivirus such as equine infectious anemia virus (EIAV).

### Marker Components and Hetero-Dimeric Markers

Each vector of the kit of the present invention comprises a nucleic acid sequence encoding a marker component. The first vector comprises a nucleic acid sequence encoding a first marker component and the second vector comprises a nucleic acid sequence encoding a second marker component.

The marker component may be unstable and unable to be expressed at the cell surface by the transduced host cell without association to a second reciprocal marker component expressed from a separate vector. The reciprocal marker component may also be unstable without association.

Upon association, the first marker component and the second marker component (reciprocal to the first marker) form a stable heteromultimeric complex which is expressed at the cell surface of the host cell.

Below are examples of first and second marker components that associate to form a stable and detectable hetero-dimeric marker capable of cell surface expression. In the absence of association the first and second marker components are unstable and do not get expressed at the surface of the cell.

### CD79a/CD79b

CD79 (cluster of differentiation 79) is protein that forms a complex with a B cell receptor and generates a signal following recognition of an antigen.

CD79 is composed of two distinct chains called CD79a and CD79b (formerly known as Ig-alpha and Ig-beta); these form a heterodimer on the surface of a B-cell stabilized by disulphide bonds. CD79a (UniProt: P11912) and CD79b (UniProt: P40259) are both members of the immunoglobulin superfamily.

Both CD79 chains contain an immunoreceptor tyrosine-based activation motif (ITAM) in their intracellular tails that they use to propagate a signal in a B cell, in a similar manner to CD3-generated signal transduction observed during T cell receptor activation on T cells.

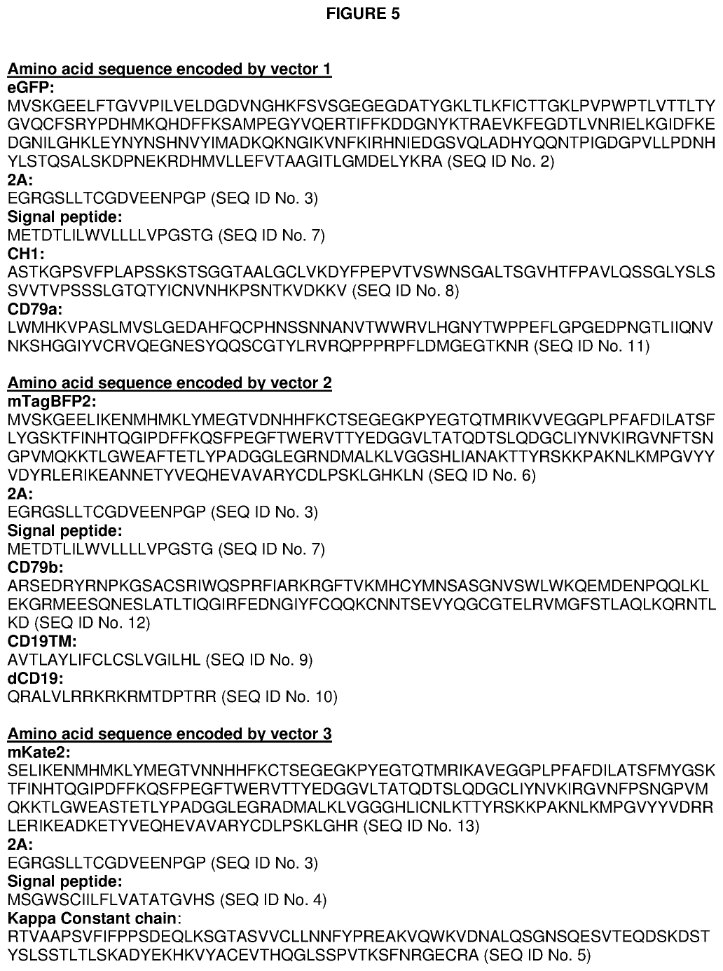

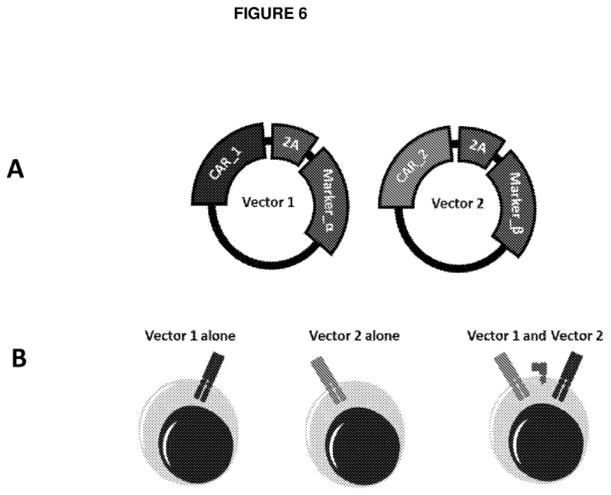

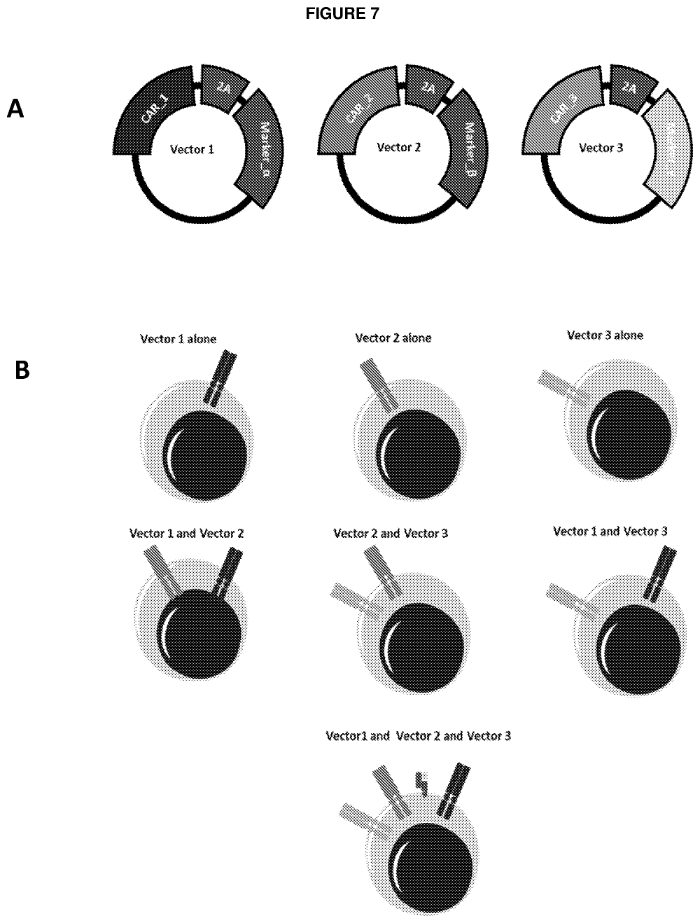

A marker component may comprise the ectodomain from CD79a or CD79b. The amino acid sequences for these domains are given in FIG. 5. A kit of vectors may comprise one vector encoding a marker component which comprises the ectodomain of CD79a and another vector encoding a marker component which comprises the ectodomain of CD79b. One or other marker may be membrane-bound, for example by having a transmembrane sequence.

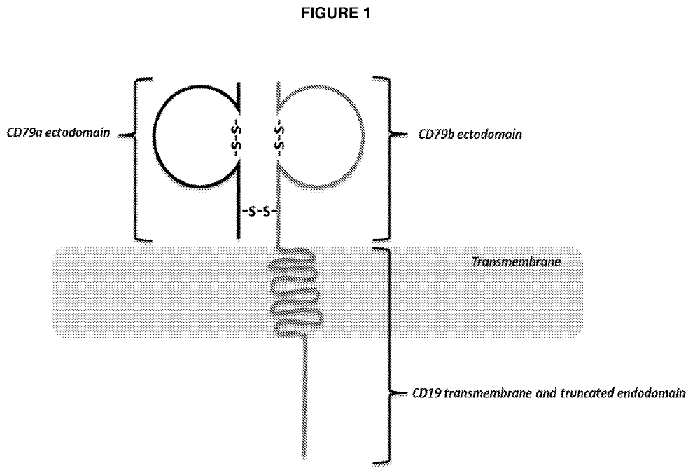

A hetero-dimeric marker arrangement is shown in FIG. 1, where the first marker component comprises a CD79a ectodomain, and the second marker component comprises a CD79b ectodomain fused to a transmembrane domain.

Upon successful cell transduction with both vectors, the CD79a ectodomain marker component and the CD79b ectodomain marker component (comprising a CD19 transmembrane domain) marker are expressed and associate, forming a stable hetero-dimeric marker which is expressed on the surface of the cell.

If either the CD79a marker component or the CD79b marker component are expressed alone in the cell, they are unstable and not expressed at the cell surface.

CH1 from IgG1/Kappa Constant Domain IgG1

IgG antibodies are multi-domain proteins with complex inter-domain interactions. Human IgG heavy chains associate with light chains to form mature antibodies capable of binding antigen. Light chains may be of the Kappa or gamma isotype.

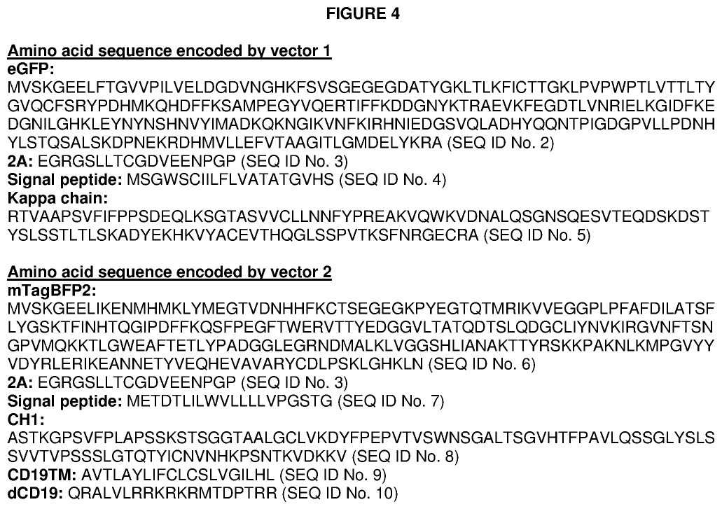

The association of heavy and light constant domains forms a stable heterodimer. A marker component may comprise a heavy chain or a light chain constant region. The amino acid sequences for a Kappa chain constant region and a CH1 region from IgG1 are given in FIG. 4, but many other suitable sequences from other antibodies are known.

A kit of vectors may comprise one vector encoding a marker component which comprises a heavy chain constant region and another vector encoding a marker component which comprises a light chain constant region. One or other marker may be membrane-bound, for example by having a transmembrane sequence.

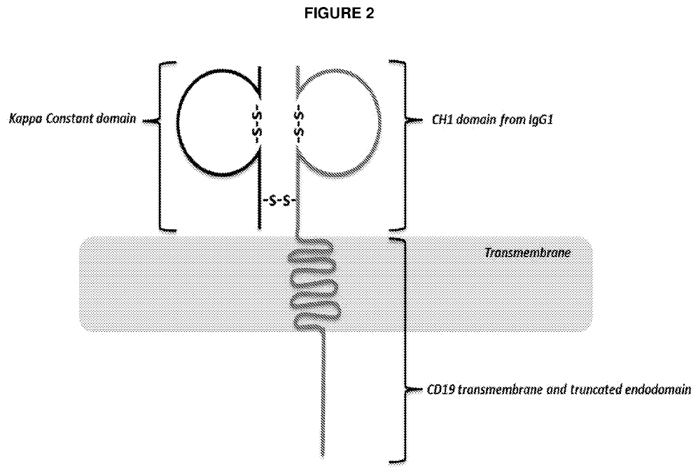

A hetero-dimeric marker arrangement is shown in FIG. 2, where the first marker component comprises a Kappa constant domain and the second marker component comprises a nucleic acid sequence encoding a CH1 domain from IgG1 fused to a transmembrane domain.

Upon successful cell transduction with both vectors, the Kappa constant domain marker component and the CH1 marker component (comprising a CD19 transmembrane domain) marker are expressed and associate, forming a stable hetero-dimeric marker which is expressed on the surface of the cell.

If either the Kappa constant domain marker component or the CH1 marker component are expressed alone in the cell, they are unstable and not expressed at the cell surface.

Table 1 provides a non-limiting list of first and second marker components, including additional marker pairs not described above. The first and second marker component pairs below may spontaneously associate to form a hetero-dimeric marker of the present invention:

TABLE 1

| First marker component | Second marker component |
|---|---|
| CD79a (UniProt: P11912) | CD79b (UniProt: P40259) |
| Kappa Constant domain | CH1 from IgG1 |
| TRDC (UniProt: B7Z8K6) | TRGC (UniProt: P03986 or P03986) |
| CD1A (UniProt: P06126) | Beta-2-microglobulin (UniProt: P61760) |
| TRBC | TRAC |

### Hetero-Trimeric Markers

The kit of vectors may comprise a first, second and third vector which encode first, second and third marker components, respectively.

One of the marker components may be membrane-bound, for example by having a transmembrane domain.

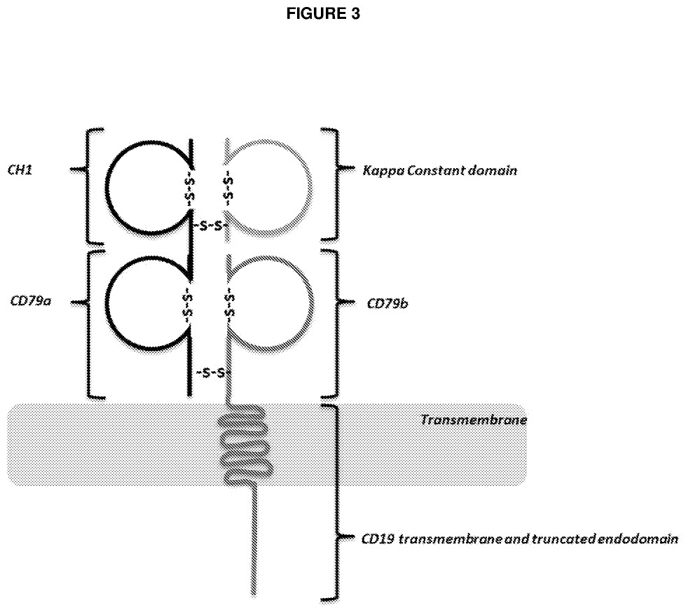

A hetero-trimeric marker arrangement is shown in FIG. 3. In this arrangement, the first marker component comprises a Kappa constant domain, the second marker component comprises a CD79b ectodomain with a CD19 transmembrane domain, and the third marker component comprises a CD79a ectodomain fused to a CH1 domain from IgG1 domain.

The Kappa constant domain marker on the first marker component associates with the CH1 domain from IgG1 on the third marker component. The CD79a ectodomain of the third marker component associates with the CD79b ectodomain on the second marker component. When all three marker components are expressed in a cell a stable heterotrimeric complex is formed which is detectable at the cell surface with an agent which recognises the Kappa constant domain. When only one or any two of the marker components are expressed in the cell, no complex is formed at the cell surface which is detectable with an agent which recignises the kappa constant domain.

The hetero-trimeric marker may be formed from any two spontaneously associating pairs of markers such as the markers described in Table 1. Formation of the hetero-trimeric marker is not limited to the pairs described above, and other spontaneously associating markers are envisioned.

This arrangement effectively permits detection of three marker components on three separate vectors by virtue of the detection of a single hetero-trimeric marker.

Similar arrangements are possible for a hetero-multimeric markers having more than three marker components. For example, the heteromultimeric marker may comprise 4, 5 or more marker components. The heteromultimeric marker may be detectable by an agent which recognises one marker component which only forms part of the heteromultimeric marker when all marker components are expressed in the cell.

### Soluble, Secreted and Membrane-Bound Markers

A marker component may be soluble when expressed alone, in the sense that it is able to diffuse freely in the cytosol of the cell.

A marker component may be secreted, in the sense that when it is expressed by a cell in the absence of other marker components it is secreted by the cell.

A marker component may membrane-bound, in the sense that is effectively anchored to a membrane.

A membrane-bound marker may, for example, comprise a transmembrane domain a stop transfer sequence, a GPI anchor or a myristoylation/prenylation/palmitoylation site.

A transmembrane domain may be derived from a protein in the marker component (for example the transmembrane domain of CD79a or CD79b), or a sequence encoding a transmembrane domain may be engineered into vector encoding the marker component.

### Detectable Hetero-Multimeric Marker

In a first aspect, the present invention provides a kit of vectors, each encoding a marker component. When a cell is transduced with all of the vectors of the kit, the expressed marker components associate to form a detectable hetero-multimeric marker.

In a second aspect, the present invention provides a detectable hetero-multimeric marker for use in detecting a transduced cell population, wherein the hetero-multimeric marker comprises at least two marker components which associate.

The hetero-multimeric marker comprises at least two marker components, but may comprise three, four or more marker components, which associate together to form a stable, detectable hetero-multimeric marker. One or more of the marker components which form the hetero-multimeric marker may be unstable when not associated together. For example, a marker component comprising the ectodomain of CD79a and a marker component comprising the ectodomain of CD79b may be unstable when the CD79a and CD79b domains are not associated together.

The term associate or association or associated is synonymous with dimerize, dimerization or dimerized, and/or bind, binding or bound. The association may form covalent bonds such as di-sulphide bonds between one marker and the other marker.

The hetero-multimeric marker may comprises multiple marker components, which unless associated with all remaining other marker components of the hetero-multimeric marker are incapable of cell surface expression.

Alternatively, one or some of the marker component(s) encoded by the kit may be capable of cell surface expression alone, whereas one marker component is only expressed at the cell surface when associated with the other marker(s). In this case, using an agent which specifically recognises this latter marker component indicates that the cell has been transduced with all the vectors in the kit.

The detectable hetero-multimeric marker is detectable by use of an agent specific to any one of the marker components. The agent is specific to a marker component which is only expressed at the cell surface when co-expressed with the other marker component(s) of the kit, i.e. it is only expressed at the cell surface as part of the hetermultimeric complex.

### Agent

The heteromultimeris marker of the present invention is detectable using an agent, such as a cell detecting or cell sorting agent. For the purposes of the present invention the term “cell sorting reagent” includes agents capable of identifying cells expressing the heteromultimeric marker: it is not limited to agents capable of identifying and sorting cells expressing the heteromultimeric marker.

The agent may, for example, derive from a ligand, small molecule or antibody.

The agent may bind specifically to any one of the marker components of the hetero-multimeric marker.

The agent may bind to a soluble or secreted marker component. The agent may bind to a transmembrane marker component if it depends on the presence of the or each other marker component for stable cell surface expression.

The agent may bind specifically to the CD79a ectodomain (see the arrangement illustrated in FIG. 1).

The agent may bind specifically to the Kappa Constant domain (see the arrangements illustrated in FIGS. 2 and 3).

An example of a small molecule agent for detecting a hetero-dimeric marker of the invention is be streptavidin. In this case, the marker component to be detected may be engineered to include a StrepTag peptide. The StrepTag is a synthetic peptide consisting of eight amino acids (YSHPQFEK—SEQ ID No. 1) which may be attached to the marker component to be detected. This peptide sequence exhibits intrinsic affinity towards streptavidin.

Detection of the StepTag peptide may involve the Strep-tag® system which allows detection by affinity chromatography.

Other examples of agents for detecting a hetero-multimeric marker include:
1, Protein A which detects CH2-CH2 on the detectable marker;
2. Glutathione which detects a Glutathione S-transferases (GST) on the detectable marker; or
3. Nickel NTA, which detects a His tag on the detectable marker.

### Cell Sorting and Purification of Transduced Cell Population

The agent may be used to select or sort the transduced cell population. This may be used in the context of purifying the transduced cells before administration to a subject, e.g. for treatment of a condition or a disease.

Magnetic nanoparticles conjugated to an agent, be it an antibody, ligand or small molecule as described above, causes the cells expressing the marker to which the agent binds, to attach to the strong magnetic field. In this step, the cells attached to the nanoparticles stay on the column, while the other cells (not expressing the marker) flow through. This way, the cells can be separated positively or negatively with respect to the particular marker.

Alternatively, fluorescent-labelled agent to the marker of interest may serve for cell separation with respect to the cells expressing the hetero-multimeric marker.

### Transmembrane Domain

The transmembrane domain is the domain of a polypeptide that spans the membrane. invention marker component may comprise a transmembrane domain such that the heteromultimeric marker is membrane bound.

A transmembrane domain may be any protein structure, which is thermodynamically stable in a membrane. This is typically an alpha helix comprising of several hydrophobic residues. The transmembrane domain of any transmembrane protein can be used to supply the transmembrane portion of the invention. The presence and span of a transmembrane domain of a protein can be determined by those skilled in the art using the TMHMM algorithm (http://www.cbs.dtu.dk/services/TMHMM-2.0/). Artificially designed TM domains may also be used.

The transmembrane domain may, for example, be derived from CD19 or CD28.

### Signal Sequence

The markers encoded by the nucleic acid sequence of the invention may comprise a signal sequence so that when the marker is associated and expressed inside a cell the nascent protein is directed to the endoplasmic reticulum (ER).

The term “signal sequence” is synonymous with “signal peptide”.

A signal sequence is a short peptide, commonly 5-30 amino acids long, present at the N-terminus of the majority of newly synthesized proteins that are destined towards the secretory pathway. These proteins include those that reside either inside certain organelles (for example, the endoplasmic reticulum, golgi or endosomes), are secreted from the cell, and transmembrane proteins.

Signal sequence commonly contain a core sequence which is a long stretch of hydrophobic amino acids that has a tendency to form a single alpha-helix. The signal sequence may begin with a short positively charged stretch of amino acids, which helps to enforce proper topology of the polypeptide during translocation. At the end of the signal sequence there is typically a stretch of amino acids that is recognized and cleaved by signal peptidase. Signal peptidase may cleave either during or after completion of translocation to generate a free signal sequence and a mature protein. The free signal sequences are then digested by specific proteases.

The signal sequence is commonly positioned at the amino terminus of the molecule, although some carboxy-terminal signal peptides are known.

Signal sequences have a tripartite structure, consisting of a hydrophobic core region (h-region) flanked by an n- and c-region. The latter contains the signal peptidase (SPase) consensus cleavage site. Usually, signal sequences are cleaved off co-translationally, the resulting cleaved signal sequences are termed signal peptides.

In the signal peptide from the murine Ig kappa chain V-III region, which has the sequence: METDTLILWVLLLLVPGSTG: the n-region has the sequence METD; the h-region (shown in bold) has the sequence TLILWVLLLV; and the c-region has the sequence PGSTG.

### Mutated Signal Sequence

A mutated signal sequence may differ in its h-region from the wild-type signal sequence. One polypeptide (which has higher relative expression) has a greater number of hydrophobic amino acids in the h-region that the other polypeptide (which has lower relative expression). The signal peptide of the polypeptide with lower relative expression may comprise one or more amino acid mutations, such as substitutions or deletions, of hydrophobic amino acids in the h-region than the signal peptide of the polypeptide with lower relative expression.

The first signal peptide and the second signal peptide may have substantially the same n- and c-regions, but differ in the h-region as explained above. “Substantially the same” indicates that the n- and c-regions may be identical between the first and second signal peptide or may differ by one, two or three amino acids in the n- or c-chain, without affecting the function of the signal peptide.

The hydrophobic amino acids in the core may, for example be: Alanine (A); Valine (V); Isoleucine (I); Leucine (L); Methionine (M); Phenylalanine (P); Tyrosine (Y); or Tryptophan (W).

The hydrophobic acids mutated in order to alter signal peptide efficiency may be any from the above list, in particular: Valine (V); Isoleucine (I); Leucine (L); and Tryptophan (W).

Of the residues in the h-region, one signal peptide (for example, the altered signal peptide) may comprise at least 10%, 20%, 30%, 40% or 50% fewer hydrophobic amino acids than the other signal peptide (for example, the unaltered signal peptide).

Where the h-region comprises 5-15 amino acids, one signal peptide may comprise 1, 2, 3, 4 or 5 more hydrophobic amino acids than the other signal peptide.

The altered signal peptide may comprise 1, 2, 3, 4 or 5 amino acid deletions or substitutions of hydrophobic amino acids. Hydrophobic amino acids may be replaced with non-hydrophobic amino acids, such as hydrophilic or neutral amino acids.

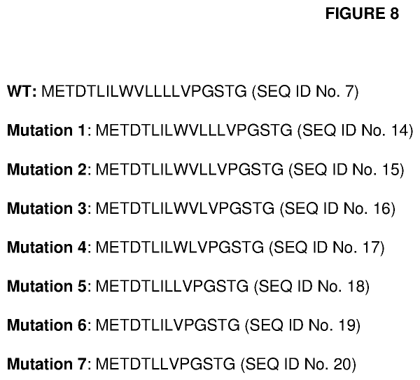

Examples of suitable mutated signal sequences for a marker component based on the signal peptide from the murine Ig kappa chain V-III region are listed in FIG. 8.

Signal sequences can be detected or predicted using software techniques (see for example, http://www.predisi.de/).

A very large number of signal sequences are known, and are available in databases. For example, http://www.signalpeptide.de lists 2109 confirmed mammalian signal peptides in its database.

### Expression Level of a First Marker and Expression Level of a Second Marker

The expression level of one marker component may be different to the expression level of another marker component. This may be achieved using one or more intracellular retention signal(s) as described in WO2016/174408, or by using alternative signal peptides as described herein and in WO2016/174409.

Where the kit of vectors encodes one or more marker component(s) which is/are soluble or secreted and a marker component which is membrane-bound, the relative expression may be taileored such that the membrane-bound marker component is expressed at a lower level than the soluble/secreted marker component(s).

Different signal peptides provide a method of controlling the expression level of one polypeptide of interest in the first vector compared another polypeptide of interest in a second vector, where the polypeptides of interest are desired to be expressed in a cell at different levels. It may be advantageous to select a transduced cell population which has a higher expression of one polypeptide sequence of interest than another.

An example of where differential expression of two polypeptides expressed in a cell is useful is where one polypeptide of interest is a chimeric antigen receptor (CAR) or engineered T cell receptor (TCR) and another polypeptide of interest is a suicide gene. Suicide genes act as an “off-switch” providing a mechanism for killing the CAR- or TCR-expressing cell, for example in the face of toxicity. A suicide gene may be more effective in killing the cell in which it is expressed if it is present in the transduced cell at a higher level than the CAR or TCR.

By altering the level of expression of maker components which are expressed on the same construct as the polypeptides of interest, it is possible to skew cell identification or sorting to transduced cells which express a higher level of a marker component which is indicative of expression of a higher level of the POI which is expressed from the same construct.

### Polypeptides of Interest (POI)

POI of the present invention may be any polypeptide that is desired to be expressed in the transduced cell population. The POI may, for example be a Chimeric antigen receptor (CAR) or an engineered T cell receptor (TCR). The POI may be a polypeptide encoding for a suicide gene.

### Cell

There is provided a cell which transduced or transfected with a kit of vectors of the present invention.

The cell may be a cytolytic immune cell such as a T cell or an NK cell.

T cells or T lymphocytes are a type of lymphocyte that play a central role in cell-mediated immunity. They can be distinguished from other lymphocytes, such as B cells and natural killer cells (NK cells), by the presence of a T-cell receptor (TCR) on the cell surface. There are various types of T cell, as summarised below.

Helper T helper cells (TH cells) assist other white blood cells in immunologic processes, including maturation of B cells into plasma cells and memory B cells, and activation of cytotoxic T cells and macrophages. TH cells express CD4 on their surface. TH cells become activated when they are presented with peptide antigens by MHC class II molecules on the surface of antigen presenting cells (APCs). These cells can differentiate into one of several subtypes, including TH1, TH2, TH3, TH17, Th9, or TFH, which secrete different cytokines to facilitate different types of immune responses.

Cytolytic T cells (TC cells, or CTLs) destroy virally infected cells and tumor cells, and are also implicated in transplant rejection. CTLs express the CD8 at their surface. These cells recognize their targets by binding to antigen associated with MHC class I, which is present on the surface of all nucleated cells. Through IL-10, adenosine and other molecules secreted by regulatory T cells, the CD8+ cells can be inactivated to an anergic state, which prevent autoimmune diseases such as experimental autoimmune encephalomyelitis.

Memory T cells are a subset of antigen-specific T cells that persist long-term after an infection has resolved. They quickly expand to large numbers of effector T cells upon re-exposure to their cognate antigen, thus providing the immune system with “memory” against past infections. Memory T cells comprise three subtypes: central memory T cells (TCM cells) and two types of effector memory T cells (TEM cells and TEMRA cells). Memory cells may be either CD4+ or CD8+. Memory T cells typically express the cell surface protein CD45RO.

Regulatory T cells (Treg cells), formerly known as suppressor T cells, are crucial for the maintenance of immunological tolerance. Their major role is to shut down T cell-mediated immunity toward the end of an immune reaction and to suppress auto-reactive T cells that escaped the process of negative selection in the thymus.

Two major classes of CD4+ Treg cells have been described—naturally occurring Treg cells and adaptive Treg cells.

Naturally occurring Treg cells (also known as CD4+CD25+FoxP3+ Treg cells) arise in the thymus and have been linked to interactions between developing T cells with both myeloid (CD11c+) and plasmacytoid (CD123+) dendritic cells that have been activated with TSLP. Naturally occurring Treg cells can be distinguished from other T cells by the presence of an intracellular molecule called FoxP3. Mutations of the FOXP3 gene can prevent regulatory T cell development, causing the fatal autoimmune disease IPEX.

Adaptive Treg cells (also known as Tr1 cells or Th3 cells) may originate during a normal immune response.

The cell may be a Natural Killer cell (or NK cell). NK cells form part of the innate immune system. NK cells provide rapid responses to innate signals from virally infected cells in an MHC independent manner

NK cells (belonging to the group of innate lymphoid cells) are defined as large granular lymphocytes (LGL) and constitute the third kind of cells differentiated from the common lymphoid progenitor generating B and T lymphocytes. NK cells are known to differentiate and mature in the bone marrow, lymph node, spleen, tonsils and thymus where they then enter into the circulation.

The cells of the invention may be any of the cell types mentioned above.

Transduced cells may either be created ex vivo either from a patient's own peripheral blood (1st party), or in the setting of a haematopoietic stem cell transplant from donor peripheral blood (2nd party), or peripheral blood from an unconnected donor (3rd party).

Alternatively, cells may be derived from ex vivo differentiation of inducible progenitor cells or embryonic progenitor cells to, for example, T or NK cells. Alternatively, an immortalized T-cell line which retains its lytic function and could act as a therapeutic may be used.

In all these embodiments, marker and POI-expressing cells are generated by introducing DNA or RNA coding for the marker and POI by one of many means including transduction with a viral vector, transfection with DNA or RNA.

The cell of the invention may be an ex vivo cell from a subject. The cell may be from a peripheral blood mononuclear cell (PBMC) sample. Such cells may be activated and/or expanded prior to being transduced with nucleic acid encoding the molecules providing the kit of vectors according to the first aspect of the invention, for example by treatment with an anti-CD3 monoclonal antibody.

The cell of the invention may be made by:
(i) isolation of a cell-containing sample from a subject or other sources listed above; and
(ii) transducing or transfecting the cell with a kit of vectors according to the first aspect of the invention.

### Pharmaceutical Composition

The present invention also relates to a pharmaceutical composition containing a plurality of cells according to the invention.

The pharmaceutical composition may additionally comprise a pharmaceutically acceptable carrier, diluent or excipient. The pharmaceutical composition may optionally comprise one or more further pharmaceutically active polypeptides and/or compounds. Such a formulation may, for example, be in a form suitable for intravenous infusion.

### Method of Treatment

The present invention provides a method for treating and/or preventing a disease which comprises the step of administering the cells of the present invention (for example in a pharmaceutical composition as described above) to a subject.

A method for treating a disease relates to the therapeutic use of the cells of the present invention. Herein the cells may be administered to a subject having an existing disease or condition in order to lessen, reduce or improve at least one symptom associated with the disease and/or to slow down, reduce or block the progression of the disease.

The method for preventing a disease relates to the prophylactic use of the cells of the present invention. Herein such cells may be administered to a subject who has not yet contracted the disease and/or who is not showing any symptoms of the disease to prevent or impair the cause of the disease or to reduce or prevent development of at least one symptom associated with the disease. The subject may have a predisposition for, or be thought to be at risk of developing, the disease.

The method may involve the steps of:
I. isolation of a cell-containing sample from a subject
II. transducing or transfecting the cell-containing sample with a kit of vectors of the present invention,
III. detecting expression of the hetero-multimeric marker using an agent, thereby identifying a transduced cell population from the sample,
IV. selecting or sorting the cell population of (III) to achieve a purified subpopulation, and
V. administering the subpopulation of (IV) which express the hetero-multimeric marker to the subject.

The present invention provides a cell composition for use in treating and/or preventing a disease.

The invention also relates to the use of a pharmaceutical composition comprising a population of transduced cells as described above in the manufacture of a medicament for the treatment and/or prevention of a disease.

The disease to be treated and/or prevented by the methods of the present invention may be a cancerous disease, such as Acute lymphoblastic leukemia (ALL), chronic lymphoblastic leukemia (CLL), bladder cancer, breast cancer, colon cancer, endometrial cancer, kidney cancer (renal cell), leukaemia, lung cancer, melanoma, non-Hodgkin lymphoma, pancreatic cancer, prostate cancer and thyroid cancer.

The cells of the present invention may be capable of killing target cells, such as cancer cells. The target cell may be characterised by the presence of a tumour secreted ligand or chemokine ligand in the vicinity of the target cell. The target cell may be characterised by the presence of a soluble ligand together with the expression of a tumour-associated antigen (TAA) at the target cell surface.

The cells and pharmaceutical compositions of present invention may be for use in the treatment and/or prevention of the diseases described above.

The cells and pharmaceutical compositions of present invention may be for use in any of the methods described above.

The invention will now be further described by way of Examples, which are meant to serve to assist one of ordinary skill in the art in carrying out the invention and are not intended in any way to limit the scope of the invention.

### EXAMPLES

### Example 1: Surface Expression of a Hetero-Dimeric Marker on Double Transduced T Cells

A first vector encoding a Kappa constant domain marker component, a 2A peptide cleavage site and an enhanced green fluorescent protein (Kappa-2A-eGFP) is mixed at a 1:1 ratio with a second vector encoding a marker component which is a fusion of a CH1 domain from IgG1 with a CD19 transmembrane domain, a 2A peptide cleavage site and an mTagBFP2 gene (CH1CD19TM-2A-mTagBFP2) and used to transduce T-cells. The resulting transduced T-cells are a mixture of cells transduced with the first vector alone (eGFP positive) or the second vector alone (mTagBFP2 positive) or double transduced with both vectors (positive for eGFP and mTagBFP2).

The cell mixture is stained with an anti-Kappa antibody conjugated to APC to show that surface expression of the heterodimeric marker is only present on double transduced T-cells (for example, cells that are double positive for eGFP and mTagBFP2). The resulting stable heterodimeric marker is as depicted in FIG. 2.

The double transduced cells are purified using anti-Kappa magnetic beads and the purity is analysed by observing the percentage of cells double positive for eGFP and mTagBFP2 by flow cytometry (FACs analysis).

As a negative control, T-cells are transduced with only one vector (either the first or second vector) and stained with an anti-Kappa antibody conjugated to APC. None of the T cells are able to express the unstable Kappa marker because the marker of the single vector is unable to associate and stabilise. In the control experiment, the anti-Kappa antibody conjugated to APC is unable to bind with any of the cells since they are not able to express a marker.

### Example 2: Surface Expression of a Hetero-Trimeric Marker on Triple Transduced T Cells

A first vector, second and third vector are mixed together at a 1:1:1 ratio and used to transduce T cells. The first vector encodes a marker component which is a fusion of a Kappa constant domain and a CH1 domain from IgG1, a 2A cleavage site, and eGFP (Kappa-2A-eGFP). The second vector encodes a marker component which is a fusion of the CH1 domain from IgG1 with a CD19 transmembrane domain, a 2A cleavage site and an mTagBFP2 gene (CD79b-CD19TM-2A-mTagBFP2). The third vector encodes a marker component which is of Kappa constant domain, a 2A peptide cleavage site and mKate2 (CH1-CD79a-2A-mKate2).

The resulting transduced T-cells are a mixture of cells transduced with the first vector alone (eGFP positive), second vector alone (mTagBFP2 positive), third vector alone (mKate2) or two out of the three available vectors (for example, positive for eGFP and mTagBFP2; or mKate2 and mTagBFP2).

The mixture of T-cells are stained with an anti-Kappa antibody conjugated to APC to show that surface expression of the heterotrimeric marker is only present on triple transduced T-cells (e.g., cells that are triple positive for eGFP, mTagBFP2 and mKate2). The resulting stable heterotrimeric marker is the marker depicted in FIG. 3.

This population of triple transduced cells are then purified to form a subpopulation of cells, using anti-Kappa magnetic beads and the purity is analysed by observing the percentage of cells triple positive for eGFP, mTagBFP2 and mKate2 by flow cytometry (FACS analysis).

As a negative control, the T-cells are transduced with either one or two of the above vectors. This may be but is not limited to either the first or second vector or the first and second vectors. The control mixture is stained with an anti-Kappa antibody conjugated to APC.

In contrast to the triple transduced cells, none of the T cells are able to express the unstable Kappa marker because the markers of the single or double vector(s) are unable to fully associate and therefore unable to stabilise or express in the cell. In the control experiment, the anti-Kappa antibody conjugated to APC is unable to bind with any of the cells.

### Example 3: Preferential Selection of Cells with a Differential Expression Level of One Polypeptide Sequence Relative to Another Polypeptide Sequence

A cell sample is transduced with a kit having a first vector encoding a marker component and a chimeric antigen receptor (Kappa-2A-CAR) and second vector encoding a marker component and a suicide gene, RapCasp9 (CH1CD19TM-2A-Rapcasp9). The RapCasp9 suicide gene is described in WO2016/151315. The second vector includes a mutated signal sequence upstream of the marker component sequence e.g., CH1CD19TM. The mutated signal sequence may be any one of the suboptimal signal sequences listed in FIG. 8. The mutated signal sequence decreases the expression level of the downstream CH1CD19TM marker encoded the second vector compared to the expression level of the Kappa marker encoded by the first vector in the cell. Since the association of the first and second markers must be in a 1:1 ratio, selection is skewed towards cells which express high level of the suicide gene Rapcasp9, compared to the level of expression of the CAR. This is useful because suicide genes such as RapCasp9 can be more effective when their expression level is higher in the cell than the expression level of the CAR.

### Example 4: Surface Expression of Heterodimer and Heterotrimer Marker in 293T Cells and Primary Human T-Cells

TABLE 2

| Chain | Construct |
|---|---|
| Vector 1 | SFGmR.aCD19_HD37_LC-2A-RQR8 |
| Vector 2 | SFGmR.eGFP-2A-aCD19_HD37_Fab_H-CD28TM-41BBz |
| Vector 3 | SGF.V5-full_human_CD19ecto-9xHis.I.eBFP |
| Positive | SFGmR.aCD19_HD37_Fab_H-CD28TM-41BBz-2A-RQR8 |
| Control | |

293T cells were singly transfected with each chain of the heterodimer marker (vector 1 or vector 2) and double transfected with both (vector 1 and vector 2). See the table 2 for the vector constructs.

Transient transfection of the 293T cells was performed using GeneJuice (Millipore), with a plasmid encoding for gag-pol (pEQ-Pam3-E36), a plasmid encoding for the RD114 envelope (RDF37), and the desired retroviral transfer vector plasmid. Viral supernatant was collected at 48 hours and 72 hours and 293T cells were stained for transfection efficiency. When the co-cultures were set up, the 293T cells were counted 48 hours after transfection and plated at 1:1 ratio for another 24 hours before staining for the heterodimeric marker.

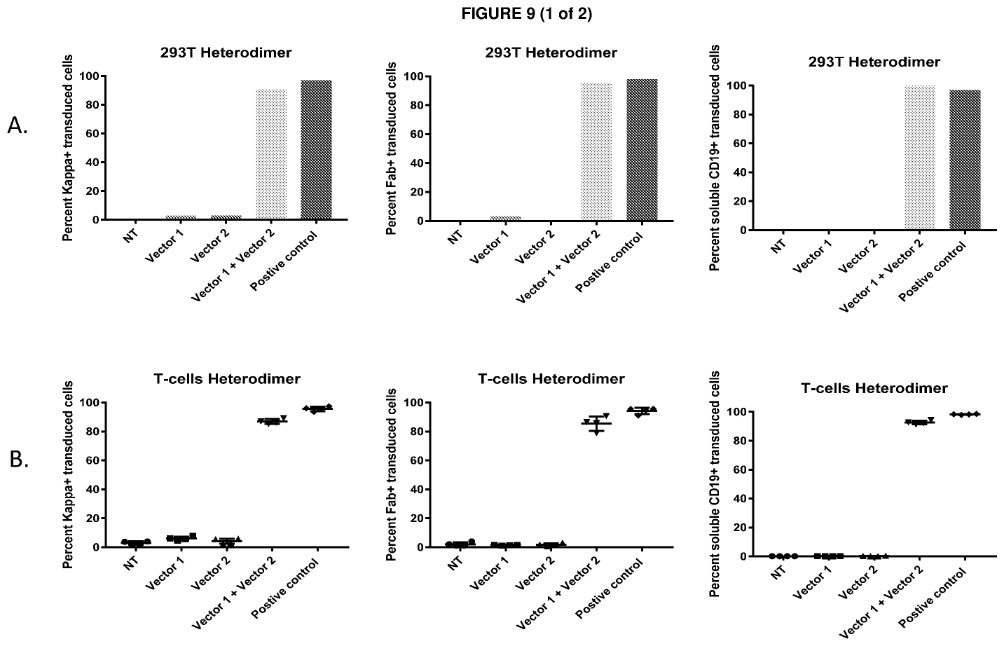

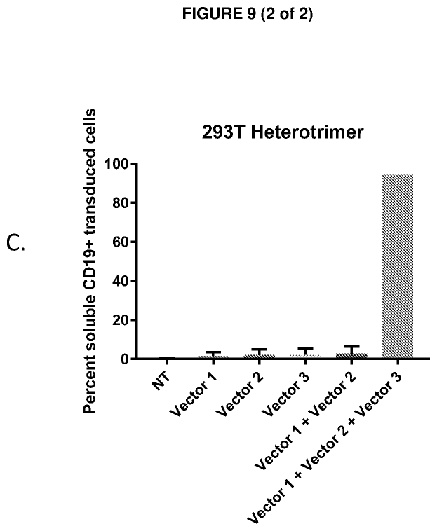

Successful assembly of the heterodimer marker in double transfected cells was assessed by flow cytometry by staining with anti-human Kappa chain antibody, anti-human Fab antibody and using soluble CD19, shown in FIG. 9A, using a standard surface stain protocol. 1×10⁵ cells were stained in round bottom 96 well plates. Surface antibodies were diluted with staining buffer (1% FBS in PBS) and added to cells at 100 μl per sample. The following antibodies were used according to manufacturer's protocol: anti-CD34 APC or PE (clone QBEnd10, R&D systems), anti-Kappa APC (BD Biosciences), anti-human Fab APC (Jackson ImmunoResearch), anti-His PE (Abcam).

A plasmid encoding for both chains of the heterodimer marker was used as a positive control.

Similarly, in primary human T-cell transduction experiments (using 4 healthy donor samples) it was demonstrated that selective expression of the heterodimer marker occurs only in double transduced T-cells with minimal background detected in single transduced T-cells. See FIG. 9B.

Peripheral blood mononuclear cells were isolated by Ficoll (GE Healthcare) gradient centrifugation and stimulated with anti-CD3/28 antibodies at 50 ng/mL. Interleukin-2 (IL-2) supplementation (100 IU/mL) was added following overnight stimulation. On day 3, T cells were harvested, plated on retronectin and retroviral supernatant, and centrifuged at 1000 g for 40 minutes. Transduction efficiency was assess 5 days later using flow cytometry, as described above.

FIG. 9C shows 293T cells singly transfected with each chain of a heterotrimer marker (vector 1, vector 2 and vector 3), double transfected (vector 1 and vector 2) and triply transfected with all three chains of the heterotrimer marker (vector 1 and vector 2 and vector 3), using the transfection method described above. The vector 1, vector 2 and vector 3 constructs as shown in table 2 were used. Successful assembly of the heterotrimer marker was assessed by staining for soluble CD19 using flow cytometry, as described above. All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

1. A kit of vectors comprising:
(i) a first vector comprising a nucleic acid sequence encoding a first marker component; and
(ii) a second vector comprising a nucleic acid sequence encoding a second marker component,
wherein, when a cell is transduced with both the first and second vectors, the first and second marker components are expressed by the cell and associate forming a hetero-multimeric marker which is recognised by a cell sorting reagent
whereas, when a cell is transduced with either the first or second vector alone, expression of the first or second marker component alone is not recognised by the cell sorting reagent.

2. A kit according to claim 1, wherein the first marker component is unstable when not associated with the second marker component, and the cell sorting reagent recognises the first marker component.

3. A kit according to claim 1, wherein both the first and second marker components are unstable when not associated, and the cell sorting reagent recognises either the first or second marker component.

4. A kit according to claim 1, wherein the first marker component is membrane-bound, and the second marker component is secreted in the absence of the first marker component and the cell sorting reagent recognises the second marker component.

5. A kit according to claim 1, wherein one marker component comprises a Kappa constant domain and the other marker component comprises the CH1 domain from IgG1.

6. A kit according to claim 1, wherein one marker component comprises a CD79a ectodomain and the other marker component comprises a CD79b ectodomain.

7. A kit according to claim 1, which comprises a third vector comprising a nucleic acid sequence encoding a third marker component
wherein, when a cell is transduced with the first, second and third vectors, the first, second and third marker components are expressed by the cell and associate forming a hetero-multimeric marker which is recognised by a cell sorting reagent;
whereas, when a cell is transduced with one or two of the first, second or third vector(s), expression of one or two of the first, second or third marker component(s) is not recognised by the cell sorting reagent.

8-10. (canceled)

11. A kit of vectors according to claim 1 wherein at least one of the vectors further comprises a nucleic acid sequence encoding a chimeric antigen receptor.

12-13. (canceled)

14. A cell-surface hetero-multimeric marker for use in detecting a transduced cell population, wherein the hetero-multimeric marker comprises at least two marker components, the first marker component encoded by a nucleic acid sequence in a first vector and the second marker component encoded by a nucleic acid sequence in a second vector, wherein the first marker and second marker components associate.

15. A hetero-multimeric marker according to claim 14, wherein the first marker and/or second marker components are unstable when not associated.

16. A hetero-multimeric marker according to claim 14, wherein the second marker component is secreted by the cell in the absence of the first marker component.

17. A cell which comprises a hetero-multimeric marker according to claim 14.

18. A cell transduced with a kit of vectors according to claim 1.

19-20. (canceled)

21. A method for making a cell according to claim 17, which comprises the step of transducing or transfecting a cell with a kit of vectors comprising:
(i) a first vector comprising a nucleic acid sequence encoding a first marker component; and
(ii) a second vector comprising a nucleic acid sequence encoding a second marker component,
wherein, when a cell is transduced with both the first and second vectors, the first and second marker components are expressed by the cell and associate forming a hetero-multimeric marker which is recognised by a cell sorting reagent
whereas, when a cell is transduced with either the first or second vector alone, expression of the first or second marker component alone is not recognised by the cell sorting reagent.

22. A method for preparing a composition of cells according to claim 17 which comprises the following steps:
(i) transducing or transfecting a cell sample with a kit of vectors comprising:
(a) a first vector comprising a nucleic acid sequence encoding a first marker component; and
(b) a second vector comprising a nucleic acid sequence encoding a second marker component,
wherein, when a cell is transduced with both the first and second vectors, the first and second marker components are expressed by the cell and associate forming a hetero-multimeric marker which is recognised by a cell sorting reagent
whereas, when a cell is transduced with either the first or second vector alone, expression of the first or second marker component alone is not recognised by the cell sorting reagent;
(ii) detecting expression of the hetero-multimeric marker using a cell-sorting reagent; and
(iii) selecting or sorting the detected cells to prepare a composition of cells which express the heteromultimeric marker.

23-25. (canceled)

26. A pharmaceutical composition comprising a plurality of cells according to claim 17.

27. (canceled)

28. A method for treating a disease, which comprises the step of administering a pharmaceutical composition according to claim 26 to a subject.

29. A method according to claim 28, which comprises the following steps:
(i) isolation of a cell-containing sample from a subject
(ii) transducing or transfecting the cell-containing sample with a kit of vectors comprising:
(a) a first vector comprising a nucleic acid sequence encoding a first marker component; and
(b) a second vector comprising a nucleic acid sequence encoding a second marker component,
wherein, when a cell is transduced with both the first and second vectors, the first and second marker components are expressed by the cell and associate forming a hetero-multimeric marker which is recognised by a cell sorting reagent
whereas, when a cell is transduced with either the first or second vector alone, expression of the first or second marker component alone is not recognised by the cell sorting reagent;
(iii) detecting expression of the hetero-multimeric marker using an cell-sorting reagent, thereby identifying a transduced/transfected cell population from the sample,
(iv) selecting or sorting the cell population of (iii) to achieve a purified subpopulation of transduced/transfected cells, and
(v) administering the subpopulation of (iv) which express the hetero-multimeric marker to the subject.

30-31. (canceled)


SEQUENCE LISTING (23 sequences)

SEQ ID NO: 1 - protein, 8 aa, Artificial Sequence
  StrepTag synthetic peptide
YSHPQFEK

SEQ ID NO: 2 - protein, 241 aa, Artificial Sequence
  eGFP sequence encoded by vector 1
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKRA

SEQ ID NO: 3 - protein, 18 aa, Artificial Sequence
  2A sequence encoded by vector 1
EGRGSLLTCGDVEENPGP

SEQ ID NO: 4 - protein, 20 aa, Artificial Sequence
  signal peptide sequence encoded by vector 1
MSGWSCIILFLVATATGVHS

SEQ ID NO: 5 - protein, 109 aa, Artificial Sequence
  Kappa chain sequence encoded by vector 1
RTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECRA

SEQ ID NO: 6 - protein, 237 aa, Artificial Sequence
  mTagBFP2 sequence encoded by vector 2
MVSKGEELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANAKTTYRSKKPAKNLKMPGVYYVDYRLERIKEANNETYVEQHEVAVARYCDLPSKLGHKLN

SEQ ID NO: 7 - protein, 20 aa, Artificial Sequence
  signal peptide sequence encoded by vector 2
METDTLILWVLLLLVPGSTG

SEQ ID NO: 8 - protein, 98 aa, Artificial Sequence
  CH1 sequence encoded by vector 2
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 9 - protein, 20 aa, Artificial Sequence
  CD19TM sequence encoded by vector 2
AVTLAYLIFCLCSLVGILHL

SEQ ID NO: 10 - protein, 19 aa, Artificial Sequence
  dCD19 sequence encoded by vector 2
QRALVLRRKRKRMTDPTRR

SEQ ID NO: 11 - protein, 111 aa, Artificial Sequence
  CD79a sequence encoded by vector 1
LWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNR

SEQ ID NO: 12 - protein, 131 aa, Artificial Sequence
  CD79b sequence encoded by vector 2
ARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKD

SEQ ID NO: 13 - protein, 230 aa, Artificial Sequence
  mKate2 sequence encoded by vector 3
SELIKENMHMKLYMEGTVNNHHFKCTSEGEGKPYEGTQTMRIKAVEGGPLPFAFDILATSFMYGSKTFINHTQGIPDFFKQSFPEGFTWERVTTYEDGGVLTATQDTSLQDGCLIYNVKIRGVNFPSNGPVMQKKTLGWEASTETLYPADGGLEGRADMALKLVGGGHLICNLKTTYRSKKPAKNLKMPGVYYVDRRLERIKEADKETYVEQHEVAVARYCDLPSKLGHR

SEQ ID NO: 14 - protein, 19 aa, Artificial Sequence
  mutated signal sequence, mutation 1
METDTLILWVLLLVPGSTG

SEQ ID NO: 15 - protein, 18 aa, Artificial Sequence
  mutated signal sequence, mutation 2
METDTLILWVLLVPGSTG

SEQ ID NO: 16 - protein, 17 aa, Artificial Sequence
  mutated signal sequence, mutation 3
METDTLILWVLVPGSTG

SEQ ID NO: 17 - protein, 16 aa, Artificial Sequence
  mutated signal sequence, mutation 4
METDTLILWLVPGSTG

SEQ ID NO: 18 - protein, 15 aa, Artificial Sequence
  mutated signal sequence, mutation 5
METDTLILLVPGSTG

SEQ ID NO: 19 - protein, 14 aa, Artificial Sequence
  mutated signal sequence, mutation 6
METDTLILVPGSTG

SEQ ID NO: 20 - protein, 13 aa, Artificial Sequence
  mutated signal sequence, mutation 7
METDTLLVPGSTG

SEQ ID NO: 21 - protein, 4 aa, Artificial Sequence
  n-region from the signal peptide sequence
METD

SEQ ID NO: 22 - protein, 10 aa, Artificial Sequence
  h-region from the signal peptide sequence
TLILWVLLLV

SEQ ID NO: 23 - protein, 5 aa, Artificial Sequence
  c-region from the signal peptide sequence
PGSTG